(19) United States
(12) Patent Application Publication
(10) Pub. No.: US 2024/0382091 A1
(43) Pub. Date: Nov. 21, 2024

(54) SYSTEM FOR MEASUREMENT OF AN INTRAOCULAR IMPLANT

(71) Applicant: Twenty Twenty Therapeutics LLC, South San Francisco, CA (US)

(72) Inventors: Oleg Rumyantsev, St. Johns, FL (US); Supriyo Sinha, Menlo Park, CA (US); Dimitri Azar, Chicago, IL (US)

(21) Appl. No.: 18/318,540

(22) Filed: May 16, 2023

(51) Int. Cl. A61B 3/16; A61B 3/00

(52) U.S. Cl. CPC A61B 3/16; A61B 3/0008

(57) ABSTRACT

An intraocular pressure sensor implant comprising a substrate defining a surface implantable in an eye; a membrane coupled to the substrate, the membrane operable to change shape as a function of an intraocular pressure of the eye and defining an active area; and a lens coupled to the substrate, the lens operable to focus an optical beam to the active area for measurement of the intraocular pressure based on a spacing between the membrane and the surface.

### FIELD

The subject matter of this disclosure relates to techniques for measuring an intraocular implant, and more specifically measuring of a pressure sensor intraocular implant with the aid of a miniature lens. Such techniques are useful for treating and/or monitoring progression of eye diseases including glaucoma, but are not limited to use with the treatment of eye disease.

### BACKGROUND

Typically, intraocular parameters within an eye, for example intraocular pressure (IOP), are measured using a tonometer. A tonometer is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky and power hungry pump arrangement that may not be practical for home use, and is not as accurate as contact tonometry. In addition, in some instances, it is desirable to be able to measure such parameters frequently and from the convenience of ones home. For example, in the case of a condition such as glaucoma, which causes chronic heightened IOP, treatment mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

A wireless, implantable, continuous IOP monitoring system has been suggested that has a commercial pressure sensing element with digital readout, and a microelectronic chip that supports wireless power/data telemetry and a wired serial communication interface with the pressure sensing element. An on-chip integrated RF coil receives power from near-field RF coupling at 915 MHz, and transmits pressure measurement bits via RF-backscattering to an external reader. To ensure accurate measurements by the external reader, however, it is critical that the external reader be precisely aligned with the pressure sensing element.

### SUMMARY

An aspect of the disclosure is a system and process for measurement of an intraocular parameter from an implant including a miniature lens to reduce alignment sensitivity. In some aspects, the intraocular parameter being measured may be the IOP of the eye. Representatively, in some aspects, a reader or sensor reading device (an electronic device that may be portable, battery powered, and held by the person themselves) is aligned with the eye, and an optical beam is emitted by a transmitter inside the reader. The reader is not physically attached to the sensor and may be outside of the eye. The beam may travel through free space (the ambient environment outside of the eye) and then enters the cornea where it impinges upon the sensor and is reflected by the sensor, towards a receiver in the reader. The reflection changes as it follows the changing IOP of the eye (for example over the course of a day). The sensor may be passive in that it does not have a source of stored power that is used to transmit a signal containing information about the IOP. Instead, a part of the sensor that is reflecting the incident beam will bend or compress, as a function of the nearby IOP, resulting in the reflection changing accordingly. An estimate of the IOP is then determined by digitally processing an electrical output signal of the receiver (that is responsive to the reflections that travelled from the sensor and then through the ambient environment before impinging on the receiver.)

Precise spatial alignment between the sensor and the reader is needed to ensure accurate measurements. For example, to perform the measurement in the previously discussed example, a single point of optical interrogation may be used in which a light beam from the handheld device is focused to an area in the center of a flexible membrane (with a spot size of ˜40 μm in diameter) of the implant. To ensure accurate measurement, the angular alignment should be ˜0.02 degrees or better between the implant and the handheld device. Such precise alignment requirements, however, may be prohibitive for a fast, reliable and easy-to-use measurement by a user. The instant disclosure therefore proposes a technique to address the above technical limitation by mounting a miniature lens with a very short focal distance directly on the sensor (implant). In some aspects, the miniature lens may have a diameter of from about 74 microns to about 125 microns, or about 100 microns. Such a miniature lens effectively constrains the optical measurement to only the area close to its focus. This also allows for the use of a collimated (as opposed to focused) light beam for the optical measurement that can greatly reduce lateral and angular alignment sensitivity. It should further be understood that the techniques proposed herein are not limited to ophthalmological/medical sensing applications and can be used in a much broader range of measurement applications involving repetitive repositioning of a miniature sensor and an external optical measurement device. For example, similar techniques may be implemented in applications involving non-contact reading of sensors or applications using spectroscopic reflectometry, a technique used to determine the thickness of thin films or the spacing between two surfaces by measuring the reflectance spectrum from the sample.

Representatively, in one aspect, an intraocular pressure sensor implant including a substrate defining a surface implantable in an eye; a membrane coupled to the substrate, the membrane operable to change shape as a function of an intraocular pressure of the eye and defining an active area; and a lens coupled to the substrate, the lens operable to focus an optical beam to the active area for measurement of the intraocular pressure based on a spacing between the membrane and the surface, is provided. In some aspects, the lens is coupled to a side of the substrate opposite the membrane. In other aspects, the lens includes a focal distance that coincides with the active area. The lens may include a convex lens and/or may be a miniature lens having a diameter less than a diameter of the optical beam. In some aspects, the optical beam includes a collimated beam. In some aspects, the lens is operable to focus the optical beam having an angular misalignment to the active area. The angular misalignment may be greater than 0.02 degrees. The substrate may be a rigid substrate and the membrane may be a flexible membrane that together define a cavity between the substrate and the eye that allows changes in the intraocular pressure to change the shape of the membrane. The optical beam may be emitted by an optical transmitter of a handheld reading device and a reflection of the optical beam from the sensor is received by the device to measure the intraocular pressure of the eye.

In other aspects, an intraocular pressure measurement system includes an intraocular pressure sensor implantable in an eye, wherein the sensor comprises a lens coupled to a first side of a substrate and a membrane coupled to a second side of the substrate that changes shape as a function of the intraocular pressure of the eye; and a sensor reading device comprising an optical transmitter to emit an optical beam that is focused by the lens to an active area of the sensor and a receiver to produce an output signal in response to receiving a reflection of the optical beam from the sensor for measuring the intraocular pressure of the eye. In some aspects, the first side of the substrate faces the sensor reading device and the second side of the substrate faces the eye when the sensor is implanted in the eye. In some aspects, the lens includes a focal point that coincides with the active area of the sensor. For example, the lens may be a convex lens. In some aspects, the lens may be operable to focus the optical beam having an angular misalignment to the active area of the sensor. In some aspects, the angular misalignment is greater than 0.02 degrees. The optical beam may include a collimated beam having a diameter greater than a diameter of the lens. The substrate may be a rigid substrate and the membrane is a flexible membrane that together define a cavity between the substrate and the eye that allows changes in the intraocular pressure to change the shape of the membrane. In some aspects, the system may further include a processor configured to estimate the intraocular pressure of the eye based on processing the output signal of the receiver. In still further aspects, the sensor reading device may be a handheld device.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows a schematic illustration of an example system including a sensor and a reader device for measuring an intraocular parameter of an eye.

FIG. 2 shows a schematic view of a user holding a reader device close to their eye for measuring an intraocular parameter of the eye of FIG. 1.

FIG. 3 shows a block diagram illustrating an example system for measuring an intraocular parameter of the eye.

FIG. 4 shows a schematic diagram of an example sensor for measuring an intraocular parameter.

FIG. 5 shows a schematic diagram of an example sensor for measuring an intraocular parameter.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

The terminology used herein is for the purpose of describing particular aspects only and is not intended to be limiting of the disclosure. Spatially relative terms, such as “beneath”, “below”, “lower”, “above”, “upper”, and the like may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (e.g., rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

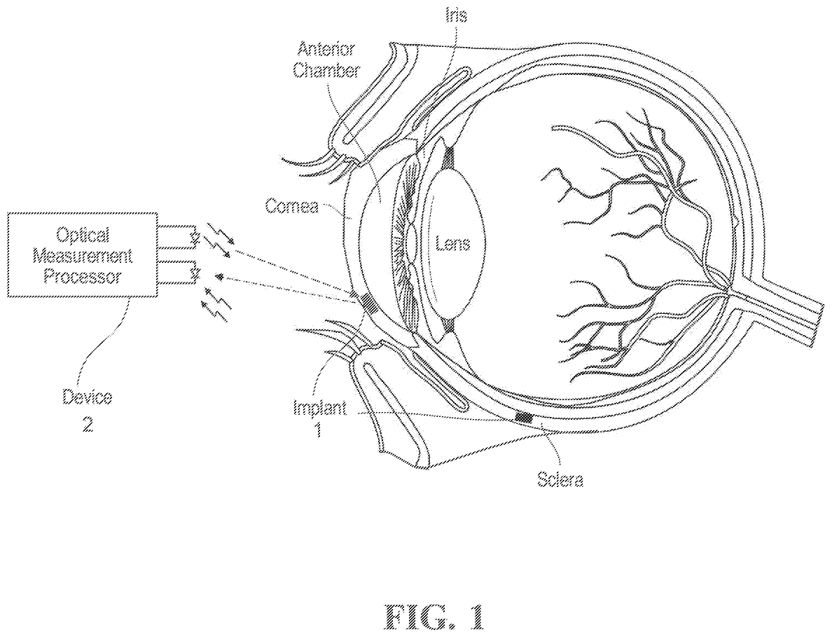

FIG. 1 shows an example system for measuring an intraocular parameter, using a handheld device 2 (e.g., a reader device) and an implant 1 (e.g., an IOP sensor). FIG. 1 shows the implant including a sensor implanted in its entirety within the cornea, or alternatively in its entirety within the sclera. While the drawings in this disclosure may not be to scale, they do illustrate that the sensor or implant 1 is small enough (e.g., about 0.25-0.5 mm thick and/or about 1 mm diameter) to be implanted into a typical cornea or sclera. Sensor 1 may be a passive sensor in that it does not have a source of stored power that is used to transmit a signal containing information about the IOP. Instead, a part of sensor 1 is designed to bend or compress or conform according to the IOP, and that part is also designed to reflect incident optical energy. As a result, sensor 1 changes how it reflects the optical energy, as a function of the IOP at that moment. Thus, the reflection changes in that it follows the changing IOP (in the anterior chamber), for example over the course of a day. As the sensor is passive, it can be made small and thin so as to be implanted into the cornea in a minimally invasive manner, more easily and with less risk of complications as compared to implant locations that are further inside the eye. The implant can be made sufficiently small, e.g. having a footprint or area in the x-y plane of about 1 mm² and a thickness in the z direction of 0.2 to 0.3 mm) so that it is entirely embedded in the cornea or sclera as shown.

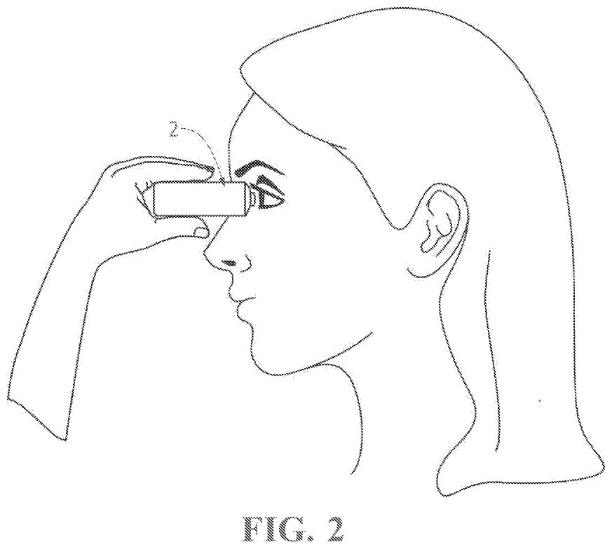

FIG. 2 illustrates a user holding the device 2 in their hand, and who has brought the handheld device close to their eye to read sensor 1. In this aspect, the handheld device 2 is a portable or handheld device configured to read the measured intraocular data once the handheld device 2 is close to the eye and aligned with sensor 1. For example, the handheld device 2 may be deemed aligned with implant 1 when device 2 determines that certain alignment parameters are met and/or alignment operations are performed (e.g., visual targets are aligned), and close to the eye when it can read sensor 1. Once device 2 determines that alignment and distance parameters are met, device 2 can then read the measured pressure data from sensor 1 and inform the user about the measured intraocular parameter.

Representatively, in some aspects, device 2 may have an optical lens that focuses the incident optical beam 9 being emitted by the transmitter 13 to an active area of sensor 1 used for measurement. The transmitter 13 may be a single photo-emitter, or it may be an array of light emitters. In some aspects, emitted beam 9 may be directional, having a narrow or directional primary lobe aimed at sensor 1. In other aspects, emitted beam 9 may be a collimated beam having multiple parallel light rays. The receiver 14 may be a single photo-detector, or it may be a one-dimensional or two-dimensional array of photo-detectors (the latter being especially useful for the case where the processor is determining the interference pattern via for example an imaging function being performed upon the signals produced by the array of photo-detectors.) The IOP is then measured by detecting changes in the cornea that cause the implanted sensor to bend or compress, during reflection of the incident beam that is detected by the receiver.

As previously discussed, to ensure an accurate reading, the focused or narrowed incident beam should be aligned to the active area of sensor 1 with an angular alignment of about 0.02 degrees or better. Such precise alignment constraints can be difficult to achieve by a user. To reduce these alignment constraints, sensor 1 may include a miniature optical lens that is configured to focus, for example, a relatively large beam or a narrow beam outside the desired angular alignment range, toward the active area of sensor 1. This, in turn, reduces lateral and angular alignment sensitivity of device 2 relative to sensor 1 allowing for faster, more reliable and easier to use measurements.

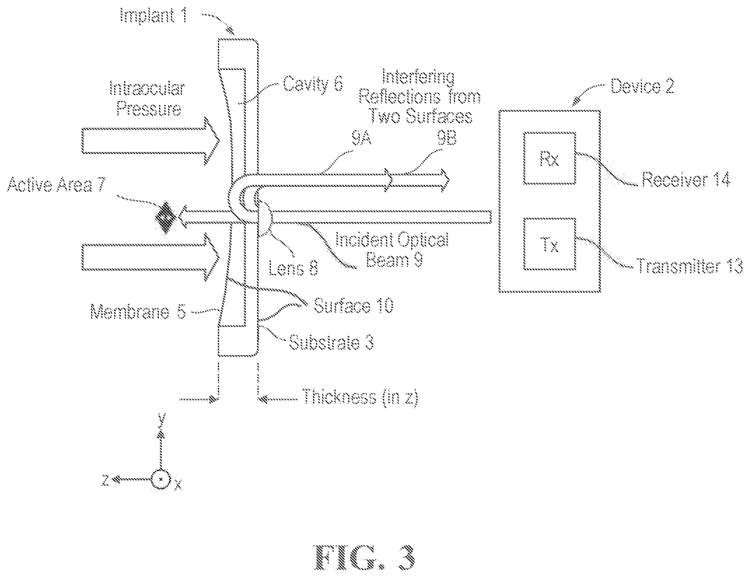

Representatively, FIG. 3 illustrates a general overview of sensor 1 and device 2. Sensor 1 may include a rigid substrate 3 to which a flexible membrane 5 (the terms rigid and flexible being relative to one another) is attached, so as to form a sealed cavity 6. Sealed cavity 6 may be bound by (or defined by) the internal faces of substrate 3 and membrane 5. Membrane 5 may be spaced from a rigid surface 10 of substrate 3 in the thickness or z-direction for example between 5 to 30 microns. Membrane 5 may be made of or coated with one or more of the following biocompatible materials: silica or any material that has a suitable difference in optical index of refraction relative to that of the surrounding tissue. Substrate 3 may be made or coated with any one of similar biocompatible materials as well. The indices of refraction of the membrane and substrate surface should be chosen to maximize the contrast of the interference that is due to reflections from those surfaces of the sensor 1.

To make IOP measurements, reader 2 contains optical transmitter (Tx) 13 and optical receiver (Rx) 14, both of which may be integrated within a housing of the reader 2. Reader 2 is aligned with the eye (as shown in FIG. 2) so that optical beam 9 of incident optical energy (radiation or waves) emitted by the transmitter 13 impinges on the active area 7 of sensor 1, while reflections 9A, 9B of that radiation from the sensor 1 are detected by the receiver 14 (as its output signal.) Note the term “beam” is used generically here, and does not require a beamforming transmitter array. Beam 9 enters the cornea where it impinges (or is incident) upon active area 7 of sensor 1, and is reflected by the sensor 1 towards the receiver. To help focus beam 9 on the active area 7 of sensor 1, sensor 1 may further include optical lens 8 arranged along substrate 5 as shown. In some aspects, lens 8 may be a convex lens, or other type of lens, having a short focal distance into sensor 1 so that the focus (focal point or focal length) of lens 8 coincides with a center of active area 7. For example, lens 8 may be a miniature lens mounted to a side of substrate 5 opposite membrane 5. For example, lens 8 may be mounted to a side of substrate 5 that faces away from the eye, or toward beam 9 emitted by device 2 (e.g., outer surface 10), and focus beam 9 through sensor to active area 7.

Representatively, in the particular example of FIG. 3, sensor 1 is oriented so that the incident beam 9 travels through lens 8 first, which in turn, focuses or aligns beam 9 with the active area 7. Beam 9 impinges the rigid substrate 3, where some of the incident energy is reflected toward the receiver 14 and some is transmitted into the cavity 6 where it is reflected (toward the receiver) off the membrane 5 as shown. An estimate of the IOP is then determined by an optical measurement processor, by digitally processing an electrical output signal of the receiver that is responsive to the interfering reflections from at least two surfaces of sensor 1. This is possible because the output signal of the receiver changes, in a detectable manner, as sensor 1 bends or changes shape due to the changing IOP.

Sealed cavity 6 may be a gaseous volume that is made to be at a low enough pressure, for example on the order of the atmosphere (atm) or lower, that allows changes in the IOP to sufficiently bend or change the shape of the membrane 5 (via movement of the corneal tissue that surrounds sensor 1 and that is caused by the changing IOP) so as to be detectable in the reflections. When sensor 1 is implanted in the orientation shown, namely that an outside surface of the membrane 5 faces the inside of the eye (or faces the anterior chamber of the eye) while the outside surface (e.g., surface 10) of the substrate 3 faces the transmitter Tx (or faces the outside environment of the eye), the incident beam 9 will be reflected at least four times including at a boundary between tissue and substrate, a boundary between substrate and cavity, a boundary between cavity and membrane and finally a boundary between the membrane and tissue. Typically, the two reflections at the cavity boundaries are what are most important and the other two reflections may be minimized with antireflection coatings or with appropriate choice of material. More generally, membrane 5 changes shape as a function of IOP, and so changes the distance between two reflection boundaries. The system further includes a processor that analyzes the signal from the receiver Rx to interpret the reflections from the sensor 1 into an estimate of the IOP, e.g., in units of mmHg. The processor may look for a spectral frequency dependent reflectivity characteristic in the receiver output signal, which can be correlated to how much the sensor 1 is being bent or compressed (by the IOP.) As such the processor may operate as a spectrometer (that performs a spectroscopy algorithm.) In another aspect, the processor analyzes the signal from the receiver Rx in a spatial sense, to determine or evaluate an interference pattern that is produced by the reflections (where the interference pattern changes as a function of bending of the membrane.) The processor may determine an absolute pressure reading as the pressure that is exerted on the sensor. To determine the pressure in the eye relative to the ambient pressure (which is typically what is needed for IOP), an ambient pressure sensor could be used in the reader and this reading can be subtracted from the absolute pressure reading. In still further aspects, the reader may also include an ambient temperature monitor to calibrate for any temperature dependent performance of the implant. In addition, the reader may include accelerometers to determine the orientation of the implant.

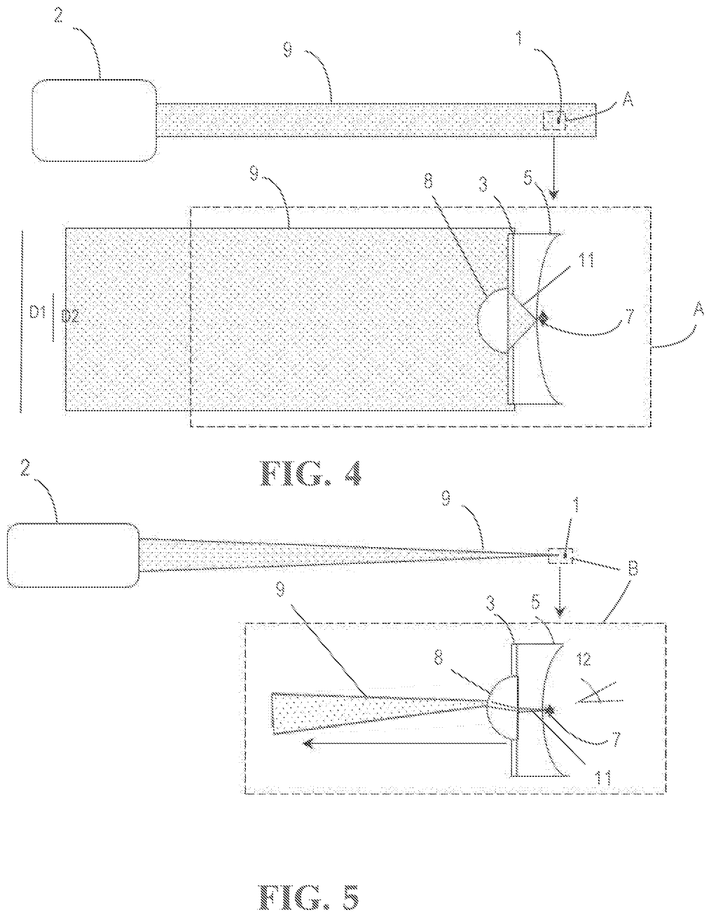

As previously discussed, however, a tilt from the normal of the optical axis or beam emitted by reader 2 to the implant's normal by ˜0.02 degrees or more could result in the reflected beam from sensor 1 possibly missing reader 2. For example, a lateral displacement of sensor 1 in any direction results in displacement of the beam focus from the active area 7 of senor 1 and an incorrect measurement of thickness. Moreover, it could be difficult for the user to know that the alignment is close to optimal. To reduce alignment sensitivity, lens 8 is designed to focus a less focused, collimated, misaligned, laterally offset or otherwise not precisely aligned beam with the active area of sensor 1 as illustrated in more detail by the magnified views of FIG. 4 and FIG. 5.

Representatively, FIG. 4 illustrates lens 8 mounted on sensor 1 to focus a portion of a collimated light beam 9 onto the active area 7. For example, as illustrated by the magnified view of section A of FIG. 4, lens 8 has a focal distance or length 11 such that the focus or focal point is directly on active area 7. For example, lens 8 may have a short focal distance into sensor 1 so that the focus of lens 8 coincides with the center of the active area 7 used for measurement. In this aspect, lens 8 can focus beam 9 having a diameter (D1) several times larger than the size or diameter (D2) of lens 8 to active area 7. For example, the diameter (D2) of lens 8 may be from about 75 microns to about 125 microns, and the diameter (D1) of beam 9 may be several times greater than the diameter (D2) of lens 8. The illustrated configuration provides two major advantages. First, lens 8 remains illuminated even if some lateral displacement of sensor/reader is introduced. Depending on the application, the user can therefore trade-off the fraction of lost optical power and sensitivity to lateral displacement. Second, any angular misalignment between reader 2 and sensor 1 results in minimal displacement of focus. In general, such displacement is equal to f*tan (a), where f is the focal length of a miniature lens and a is angular misalignment. For f=200 microns (μm) and active area of 40 μm, this results in acceptable angular misalignment of +6 degrees (°). Depending on the application, the user can therefore trade-off the fraction of captured optical power (proportional ratio of the area of the lens to the area of the beam cross-section) and angular/lateral displacement sensitivity (proportional to the ratio of miniature lens focal length and the size of the active area).

FIG. 5 illustrates another scenario in which lens 8 mounted on sensor 1 is used to focus a misaligned beam 9 onto the active area 7. For example, as illustrated by the magnified view of section B of FIG. 5, beam 9 may be a relatively narrow or focused beam, however, may be misaligned or otherwise offset of tilted by an angle 12 (e.g., about 0.02 degrees or greater) from the normal or center of active area 7 of sensor 1. In the absence of lens 8, this misalignment could result in beam 9 missing active area 7 of sensor 1 and an incorrect measurement of thickness. As can be seen from section B of FIG. 5, however, when beam 9 hits lens 8, lens 8 re-directs the misaligned beam 9 focusing it to active area 7. In this aspect, lateral and/or angular alignment sensitivity is significantly reduced making the system more reliable and easier to use.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, it should be understood that while a passive implant or sensor is primarily disclosed herein, in some aspects, the sensor or implant may be considered an active sensor and have a source of stored power that is used to transmit a signal containing information about the IOP. In addition, the aspects disclosed herein are not limited to ophthalmological/medical sensing applications and can be used in a much broader range of measurement applications involving repetitive repositioning of the miniature sensor and an external optical measurement device. The description is thus to be regarded as illustrative instead of limiting.

What is claimed is:

1. An intraocular pressure sensor implant comprising:
a substrate defining a surface implantable in an eye;
a membrane coupled to the substrate, the membrane operable to change shape as a function of an intraocular pressure of the eye and defining an active area; and
a lens coupled to the substrate, the lens operable to focus an optical beam to the active area for measurement of the intraocular pressure based on a spacing between the membrane and the surface.

2. The implant of claim 1 wherein the lens is coupled to a side of the substrate opposite the membrane.

3. The implant of claim 1 wherein the lens comprises a focal length that coincides with the active area.

4. The implant of claim 1 wherein the lens comprises a convex lens.

5. The implant of claim 1 wherein the lens comprises a miniature lens having a diameter less than a diameter of the optical beam.

6. The implant of claim 1 wherein the optical beam comprises a collimated beam.

7. The implant of claim 1 wherein the lens is operable to focus the optical beam having an angular misalignment to the active area.

8. The implant of claim 7 wherein the angular misalignment is greater than 0.02 degrees.

9. The implant of claim 1 wherein the substrate is a rigid substrate and the membrane is a flexible membrane that together define a cavity between the substrate and the eye that allows changes in the intraocular pressure to change the shape of the membrane.

10. The implant of claim 1 wherein the optical beam is emitted by an optical transmitter of a handheld reading device and a reflection of the optical beam from the sensor is received by the device to measure the intraocular pressure of the eye.

11. An intraocular pressure measurement system comprising:
an intraocular pressure sensor implantable in an eye, wherein the sensor comprises a lens coupled to a first side of a substrate and a membrane coupled to a second side of the substrate that changes shape as a function of the intraocular pressure of the eye; and
a sensor reading device comprising an optical transmitter to emit an optical beam that is focused by the lens to an active area of the sensor and a receiver to produce an output signal in response to receiving a reflection of the optical beam from the sensor for measuring the intraocular pressure of the eye.

12. The system of claim 11 wherein the first side of the substrate faces the sensor reading device and the second side of the substrate faces the eye when the sensor is implanted in the eye.

13. The system of claim 11 wherein the lens comprises a focal point that coincides with a center of the active area of the sensor.

14. The system of claim 11 wherein the lens comprises a convex lens.

15. The system of claim 11 wherein the lens is operable to focus the optical beam having an angular misalignment to the active area of the sensor.

16. The system of claim 15 wherein the angular misalignment is greater than 0.02 degrees.

17. The system of claim 11 wherein the optical beam comprises a collimated beam having a diameter greater than a diameter of the lens.

18. The system of claim 11 wherein the substrate is a rigid substrate and the membrane is a flexible membrane that together define a cavity between the substrate and the eye that allows changes in the intraocular pressure to change the shape of the membrane.

19. The system of claim 11 further comprising a processor configured to estimate the intraocular pressure of the eye based on processing the output signal of the receiver.

20. The system of claim 11 wherein the sensor reading device is a handheld device.
